(19) United States
(12) Patent Application Publication
(10) Pub. No.: US 2023/0245768 A1
(43) Pub. Date: Aug. 3, 2023

(54) FAMILY VENTILATOR DASHBOARD FOR MEDICAL VENTILATOR

(71) Applicant: Covidien LP, Mansfield, MA (US)

(72) Inventors: Patrick J. Nellis, Georgetown (CA); Michal Marach, Broomfield, CO (US); Gary S Milne, Louisville, CO (US)

(73) Assignee: Covidien LP, Mansfield, MA (US)

(21) Appl. No.: 18/158,026

(22) Filed: Jan. 23, 2023

Related U.S. Application Data
(60) Provisional application No. 63/267,442, filed on Feb. 2, 2022.

(51) Int. Cl. G16H 40/63; G06F 3/04847; G06F 3/0482; G06T 13/00; A61M 16/00

(52) U.S. Cl. CPC G16H 40/63; A61M 16/024; G06F 3/0482; G06F 3/04847; G06T 13/00; A61M 2205/502; A61M 2205/6063; G06T 2200/24

(57) ABSTRACT

A ventilator for generating a family dashboard for a patient being ventilated by the ventilator. The ventilator includes a pressure generating system for generating pressurized breathing gases for delivery to the patient and a display coupled to the pressure generating system. The ventilator performs operations including displaying, on the display, a set of ventilation waveforms; detecting a triggering condition for a family dashboard; and based on detecting the triggering condition, removing the ventilation waveforms from the display and displaying, on the display, the family dashboard, wherein the family dashboard includes at least a total ventilator support indicator that indicates an amount of total ventilator support being provided to the patient.

### CROSS REFERENCE TO RELATED APPLICATIONS

This application claims the benefit of U.S. Provisional Application No. 63/267,442 filed Feb. 2, 2022, entitled “Family Ventilator Dashboard for Medical Ventilator,” which is incorporated herein by reference in its entirety.

### INTRODUCTION

Medical ventilator systems are used to provide ventilatory and supplemental oxygen support to patients. These ventilators typically comprise a connection for pressurized gas (air, oxygen) that is delivered to the patient through a conduit or tubing. As the ventilators operate, a display of the ventilator may display data regarding operation of the ventilator, which may help a clinician or medical professional determine or assess if the ventilator is operating properly.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for displaying a family or patient dashboard on a display, such as a display of a ventilator. In an example, the technology relates to a ventilator for generating a family dashboard for a patient being ventilated by the ventilator. The ventilator includes a pressure generating system for generating pressurized breathing gases for delivery to the patient; a display coupled to the pressure generating system; a processor; and memory storing instructions that, when executed by the processor, cause the ventilator to perform operations. The operations include displaying, on the display, a set of ventilation waveforms; detecting a triggering condition for a family dashboard; based on detecting the triggering condition, removing the ventilation waveforms from the display and displaying, on the display, the family dashboard, wherein the family dashboard includes at least a total ventilator support indicator that indicates an amount of total ventilator support being provided to the patient.

In an example, the triggering condition is one of an expiration of a timeout timer or a selection of a family dashboard activation element. In another example, the operations further comprise, based on at least one of a patient type or patient condition, selecting the family dashboard from a set of preconfigured family dashboards. In yet another example, the total ventilator support indicator is a total ventilator support plot with an x-axis representing time and a y-axis representing the amount of total ventilator support. In still another example, the amount of total ventilator support is calculated from at least a spontaneous breathing factor and a pressure factor. In a further example, the amount of total ventilator support is further calculated from at least an oxygenation factor. In still yet another example, the amount of total ventilator support is a percentage ranging from 0% to 100% and is based on at least a total minute volume, a spontaneous minute volume, a peak inspiratory pressure, and an oxygenation factor.

In another example, the total ventilator support indicator is a current ventilator support indicator. The still another example, the family dashboard further includes at least one of: an independent breathing indicator that indicates a portion of breaths that are being triggered by the patient; or a breathing muscle strength plot that indicates a breathing muscle strength of the patient. In yet another example, the family dashboard further includes a scannable code configured to direct a scanning device to a webpage with additional information regarding a condition of the patient.

In another aspect, the technology relates to a method for generating a family dashboard for a ventilated patient. The method includes displaying, on a display of a ventilator, one or more ventilation waveforms; detecting a triggering condition for a family dashboard; based on detecting the triggering condition, removing the ventilation waveforms from the display and displaying, on the display, the family dashboard, wherein the family dashboard includes at least three user interface elements selected from: a total ventilator support indicator that indicates an amount of ventilator support being provided to the patient; an independent breathing indicator that indicates a portion of breaths that are being triggered by the patient; a breathing muscle strength plot that indicates a breathing muscle strength of the patient; patient help suggestions; a scannable code configured to direct a scanning device to a webpage with additional information regarding a condition of the patient; clinician contact information; a real-time breathing indicator; and a patient weight plot.

In an example, the family dashboard includes the total ventilator support indicator, the independent breathing indicator, and the breathing muscle strength plot. In another example, the method further includes receiving a selection to enter a configuration mode; entering the configuration mode; while in the configuration mode, receiving an input to resize or reposition one user interface element of the user interface elements in the family dashboard; and based on the received input, resizing or repositioning the user interface element. In still another example, the method further includes receiving a selection to enter a configuration mode; entering the configuration mode; while in the configuration mode, receiving a selection of an add-element indicator; in response to receiving the selection of the add-element indicator, presenting additional user interface elements for adding to the family dashboard; receiving a selection of one of the additional user interface elements; and displaying the selected additional user interface element in the family dashboard. In yet another example, the method further includes receiving a selection of one of the user interface elements of the family dashboard; and in response to receiving the selection, displaying underlying data used to generate the user interface element.

In another aspect, the technology relates to a method for generating a family dashboard for a ventilated patient. The method includes detecting, by a ventilator, a triggering condition to display a family dashboard; based on at least one of a patient type or a patient condition, selecting, by the ventilator, a family dashboard from a set of preconfigured family dashboards; and displaying, on a display of the ventilator, the selected family dashboard.

In an example, the patient type is pediatric and the selected family dashboard is a pediatric dashboard that includes a patient weight plot that indicates a weight trend of the patient. In another example, the pediatric dashboard further includes an animated lung-shaped breathing indicator. In still another example, the pediatric dashboard further includes clinician contact information and a clinician image. In yet another example, the pediatric dashboard further includes a scannable code configured to direct a scanning device to a webpage with additional information regarding a condition of the patient.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts a diagram illustrating an example of a medical ventilator connected to a human patient.

FIG. 2 depicts an example ventilator displaying complex ventilation data for a clinician.

FIG. 3A depicts a display with an example patient or family dashboard.

FIG. 3B depicts additional or alternative user-interface elements for the patient or family dashboard.

FIG. 3C depicts additional or alternative user-interface elements for the patient or family dashboard.

FIG. 4 depicts an example flow chart for generating a patient or family dashboard.

FIG. 5 depicts another example flow chart for configuring a patient or family dashboard.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

Medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

As briefly discussed above, medical ventilators may also include a display that presents data about the operation of the ventilator. The presented data may include complex graphs or plots along with specific ventilation settings. Such data is useful only to medical professionals or clinicians that have been well trained in medical ventilation and can understand the complexities of the data and its usefulness. For patients and their families, however, such complex data provides little use or insight into patient’s condition or progress. Moreover, the complex data may lead to incorrect conclusions by the patient or family members and also lead to additional questions for the medical professionals, whose time is becoming increasingly strained.

Among other things, the present technology provides a patient or family dashboard that is displayed on the display of the ventilator or on a remote device. The family dashboard presents data about the patient and the patient’s progress or trajectory in a format that can be understood by the patient and the patient’s family members. In addition, the data and metrics presented in the family dashboard may also provide benefits to the clinicians in easily assessing a patient’s condition and/or progress directly from the ventilator screen. For example, the family dashboard may include new metrics indicating the amount of total ventilator support being received by the patient and/or a trend of that ventilator support. Additionally, indicators may indicate a portion of the patient’s independent breathing and/or the patient’s strength and the respective trends or progressions of such indicators. Suggestions on how the patient and/or family member may be able to help may also be dynamically generated for display in the family dashboard.

The family dashboard may be selectively activated and/or automatically activated. For example, an input from a user (e.g., clinician) may cause the family dashboard to be displayed. In other examples, the family dashboard may automatically be displayed after timeout period from the last interaction with the ventilator by a clinician. The displayed user-interface elements of the family dashboard may also be configurable by a clinician. For instance, the clinician may be able to select which user interface elements are to be included in the family dashboard and/or how the user interface elements are arranged in the dashboard. In some examples, the user interface elements of the family dashboard may be automatically selected and/or dynamically adjusted based on patient characteristics or condition. For instance, a family dashboard for an adult patient may be configured differently than a family dashboard for a pediatric or neonatal patient.

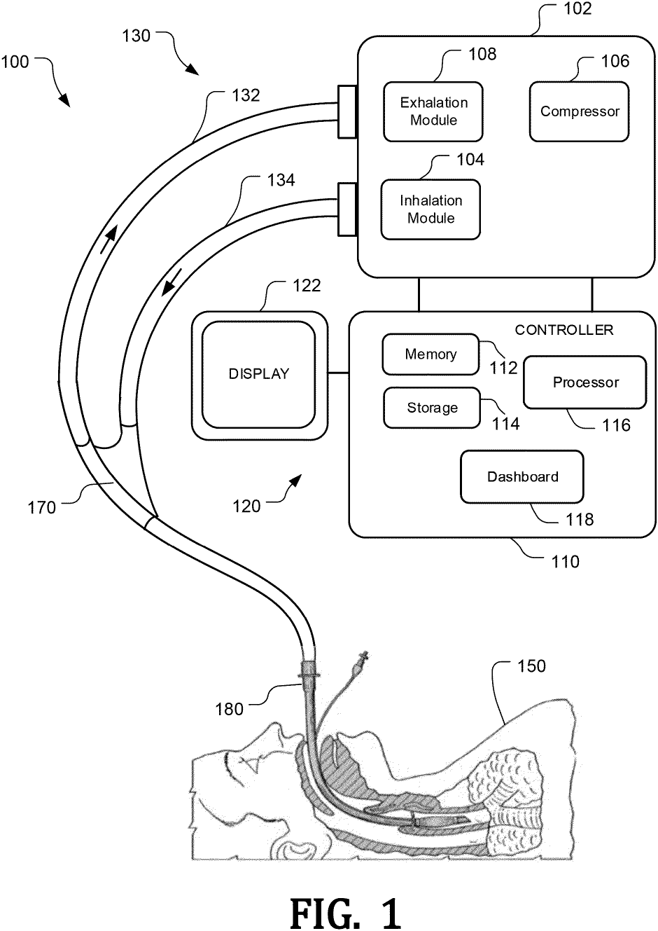

FIG. 1 is a diagram illustrating an example of a medical ventilator 100 connected to a human patient 150. The ventilator 100 may provide positive pressure ventilation to the patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator). The sensors may measure flows and/or pressures of the gases flowing into and out of the pneumatic system 102. The measurements from the sensors may be used to calculate and/or determine the volumes, pressures, and/or other properties of the breathing gases discussed herein.

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media may be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid-state memory technology, CD-ROM, DVD, or other optical storage, magnetic cassettes, magnetic tape, magnetic disk storage or other magnetic storage devices, or any other medium which can be used to store the desired information and which can be accessed by the computer. The memory 112 may store instructions that, when executed by the processor, cause the ventilator 100 to perform the operations discussed herein. For instance, the controller 110 may also include dashboard algorithms or instructions 118 for generating and managing the dashboards discussed herein.

Communication between components of the ventilator system or between the ventilator system and other therapeutic equipment, remote monitoring systems, and/or remote computing devices (tablets, mobile phones, etc.) may be conducted over a distributed network via wired or wireless means. For instance, the ventilation data and/or display data may be communicated over local area networks, wide area networks, and/or directly to another device such as through a BLUETOOTH or similar short-range communication protocol.

The display 122 presents various input screens, for receiving input, and various display screens, for presenting useful information. Inputs may be received from a clinician. The display may present user interfaces, such a graphical user interface (GUI), which may include the family dashboards discussed herein. The display 122 may be an interactive display, e.g., a touch-sensitive screen or otherwise. The presented user interfaces may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 100 may be provided, for instance by a wheel, keyboard, mouse, or other suitable interactive device.

The ventilator 100 may be controlled based on ventilation settings. Ventilation settings may include any appropriate input for configuring the ventilator to deliver breathable gases to a particular patient, including measurements and settings associated with inhalation flow and exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis/condition, ideal body weight, predicted body weight, gender, ethnicity, etc.) of the particular patient according to any appropriate standard protocol or otherwise. In some cases, certain ventilation settings may be adjusted based on measured pressures, flows, or other characteristics of the breathing gases. Ventilation settings may include inhalation flow, frequency of delivered breaths (e.g., respiratory rate, (ƒ), tidal volume (V_{T}), PEEP level, etc.).

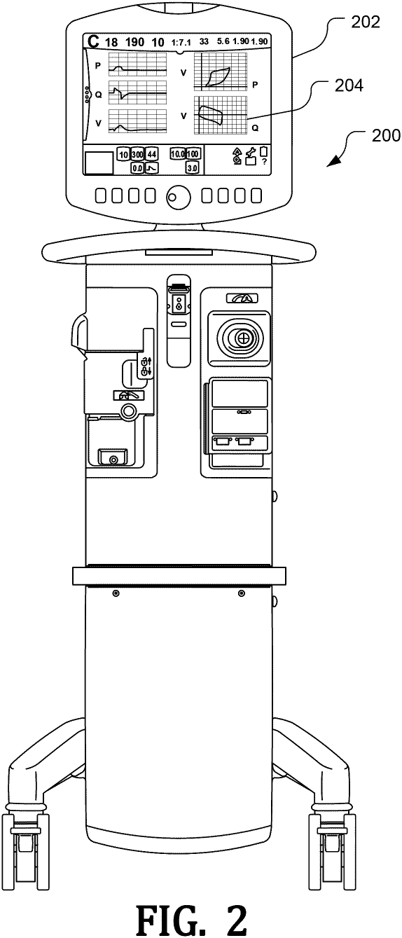

FIG. 2 depicts an example ventilator 200 displaying complex ventilation data 204 on a display 202. The complex ventilation data 204 may include the types of graphs, plots, settings, tools, etc. that are traditionally displayed on a ventilator. For example, the complex data may include ventilation waveforms such as pressure versus time plots, flow versus time plots, volume versus time plots, and volume versus pressure loops, among other types of complex representations of data. Such complex data is intended for well-trained clinicians or medical professionals that have been trained on how to interpret and utilize such data. That complex data, however, is of little use to the patient and/or the patient’s family. In some situations, a physician may also have limited use for the data unless the physician is also a respiratory therapist and/or had specific training in interpreting the complex ventilator data.

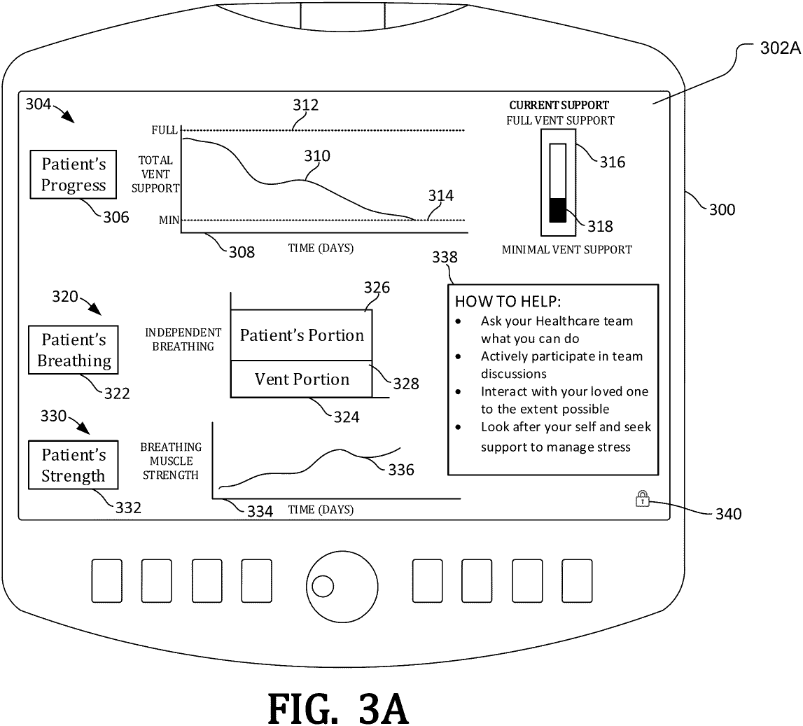

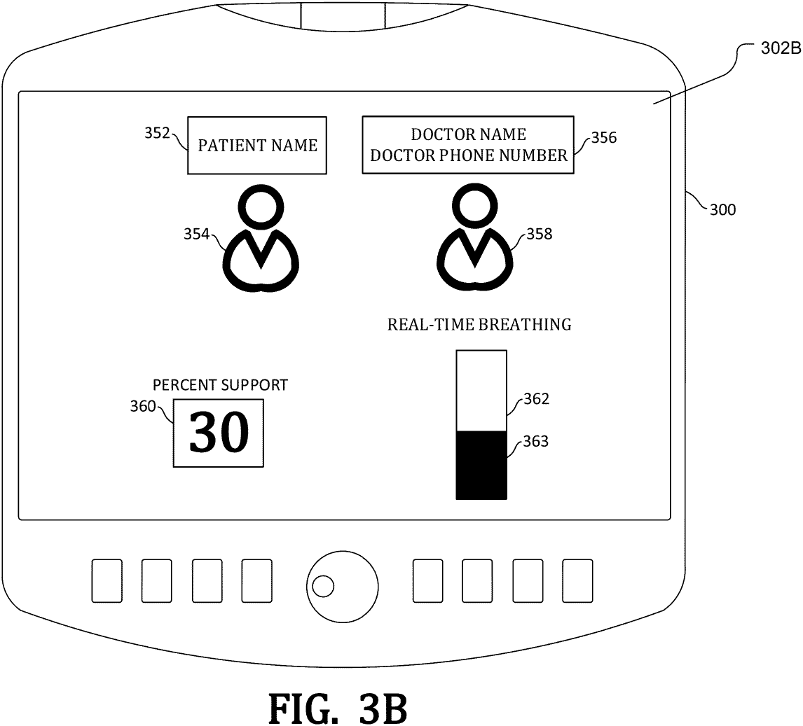

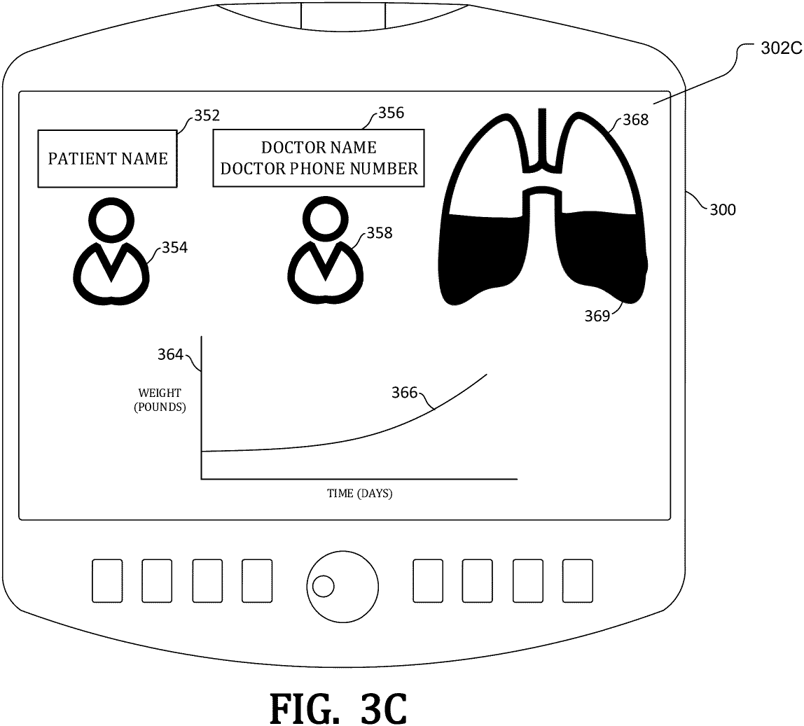

FIG. 3A depicts an example display 300 with an example patient or family dashboard 302A. The display 300 may be a display of a ventilator, such as the ventilator displays discussed above. The example family dashboard 302A is a GUI displayed on the display 300. The family dashboard 302A presents simplified data regarding the condition and/or progress of the patient that can be readily interpreted by the patient and/or the patient’s family. The family dashboard 302A may also be useful for clinicians in assessing a patient (such as during rounds) and/or explaining a patient’s condition to the patient and/or to the patient’s family members.

The family dashboard 302A may include multiple sections with multiple different types of indicators that convey information about the patient’s condition. For example, the family dashboard 302A may include a patient progress section 304 that may include a progress section title 306. While the progress section title 306 uses the term “patient” in FIG. 3A, the term “patient” in all the user interface elements, including the progress section title 306, may be replaced with the current patient’s actual name. For instance, if the ventilated patient’s name is John Smith, the progress section title 306 may read “John’s Progress.” The name of the patient may be input by a clinician during initialization of ventilation and/or during configuration of the family dashboard 302A.

The progress section 304 may include data regarding how much ventilator support the patient is receiving. For instance, the patient progress section 304 may include a total ventilator support plot 308 that indicates the amount of total ventilator support being provided to the patient over time. The x-axis of the total ventilator support plot 308 may represent time and the y-axis may represent a total ventilator support value or amount. A trend line 310 for the total ventilator support over time may be displayed in the plot 308. An upper line 312 may also be included that indicates full ventilator support, and a lower line 314 may be included that indicates minimum ventilator support 314.

The total ventilator support may be an index ranging from 0%-100%, where 0% indicates minimum ventilator support and 100% indicates full ventilator support. The total ventilator support may be defined based on factors such as spontaneous breathing, minute volume, oxygenation (e.g., fraction of inspired oxygen (FiO2)), peak inspiratory pressure (PIP), and/or positive end-expiratory pressure (PEEP), among other potential factors.

In some examples, full ventilator support may be defined as having 0% spontaneous breaths over a prior set time period (e.g, 1 minute), a PIP at 35 cmH₂0 or higher, and/or an oxygenation setting of 80% or higher. Minimum ventilator support may be defined as having a 90% or greater spontaneous breaths over the prior set time period, a PIP of 10 cmH₂0 or less, and/or an oxygenation setting of 40% or lower. The definitions of full ventilator support and minimum ventilator support may also be set to that of criteria from medical associations or research publications, such as weaning criteria from the American Association for Respiratory Care (AARC). In some examples, the definitions of full ventilator support and minimum ventilator support may also be customized by the clinician so that the respective definitions are appropriate for the specific patient and/or condition of the patient.

An example equation for calculating the total ventilator support may be as follows: TVS = F_{Spont} x F_{Pressure,} where TVS is the total ventilator support, F_{Spont} is a spontaneous breathing factor and F_{Pressure} is a pressure factor. As a specific example equation, the total ventilator support may be represented as a percentage and the equation is shown as follows in Equation 1:

$$\% T V S = \frac{{V}_{E T O T} - {V}_{E S P O N T}}{{V}_{E T O T}} \times \frac{P I {P}_{C U R R E N T}}{P I {P}_{F U L L}} \times 100$$

In the above equation, V_{E TOT} is the total minute volume, which is the volume of gas delivered to the patient per breath times the number of breaths in a minute. V_{E} _{SPONT} is the spontaneous minute volume for breaths that were triggered by the patient (e.g., spontaneous breaths). PIP_{CURRENT} is the current PIP, and PIP_{FULL} is the PIP that is used to define full ventilator support, which as discussed above, may be 35 cmH₂0 or greater. The total ventilator support may be calculated at regular intervals, and the total ventilator support plot 308 may be updated each time the total ventilator support is calculated for the patient.

In some examples, oxygenation may also be considered, and an example equation may be TVS = F_{Spont} x F_{Pressure} x F_{oxygen}, where F_{oxygen} is an oxygenation factor. Additional or alternative factors may also be used for the calculation or determination of TVS, such as a PEEP factor, a ΔP factor (where ΔP is equal to PIP - PEEP or Plateau Pressure - PEEP, known as driving pressure), a driving pressure factor, a driving pressure, a partial pressure of oxygen (PaO₂) to fractional oxygen (FiO₂) ratio factor, a ventilation-perfusion matching factor, a respiratory muscle pressure (Pmus) factor, among other possible factors.

Another example equation that may be used in calculating total ventilator support is as follows in Equation 2:

$$\begin{matrix}\% T V S = \left[\left\{\frac{{V}_{E T O T} - {V}_{E S P O N T}}{{V}_{E T O T}} + \frac{P I {P}_{C U R R E N T} - P I {P}_{M I N}}{P I {P}_{M A X} - P I {P}_{M I N}}\right)\right) \\ \left(\left(+ \frac{F i O {2}_{C U R R E N T} - F i O {2}_{M I N}}{1 - F i O {2}_{M I N}}\right\} \div 3\right] \times 100\end{matrix}$$

Equation 2 further incorporates an oxygenation factor that utilizes FiO2 levels. Specifically, the oxygenation factor in Equation 2 uses the difference of the current FiO2 setting (FiO2_{CURRENT}) and the minimum FiO2 setting (FiO2_{MIN}). The denominator of the oxygenation factor is the difference between 1 and the minimum FiO2 setting because a setting of 1 (or 100% oxygen) is the maximum FiO2 setting. Equation 2 also uses a different pressure factor that utilizes the current PIP (PIP_{CURRENT}), the minimum PIP (PIP_{MIN}), and the maximum PIP (PIP_{MAX}). The maximum PIP may correspond to the PIP_{FULL} value used above in Equation 1.

Another example equation that may be used in calculation total ventilator support is as follows in Equation 3:

$$\begin{matrix}\% T V S = \left[\left\{\frac{{V}_{E T O T} - {V}_{E S P O N T}}{{V}_{E T O T}} + \frac{Δ {P}_{C U R R E N T} - Δ {P}_{M I N}}{Δ {P}_{M A X} - Δ {P}_{M I N}} +\right)\right) \\ \left(\left(\frac{P E E {P}_{C U R R E N T} - P E E {P}_{M I N}}{P E E {P}_{M A X} - P E E {P}_{M I N}} + \frac{F i O {2}_{C U R R E N T} - F i O {2}_{M I N}}{1 - F i O {2}_{M I N}}\right\} \div 4\right] \times 100\end{matrix}$$

Equation 3 is substantially similar to Equation 2 with the exception that Equation 3 further includes a ΔP factor. ΔP is equal to PIP – PEEP or Plateau Pressure – PEEP, known as driving pressure). The ΔP factor utilizes a current ΔP value (ΔP_{CURRENT}), a minimum ΔP value (ΔP_{MIN}), and a maximum ΔP value (ΔP_{MAX}).

The maximum (or full) values and the minimum values used in the equations above may be preset by the ventilator and/or adjusted by the physician. The preset values may be based on clinical data and/or definitions or criteria from medical associations or research publications, such as weaning criteria from the American Association for Respiratory Care (AARC). In addition, while each of the factors used in the above equations are weighted equally, in other examples, the factors may be weighted differently in calculating the TVS value.

In Equations 2-3 above, in some instances the numerators of the factors may become negative when the current values are below the defined minimum values. For example, the minimum FiO2 value (FiO2_{MIN}) may be set for 40%, but the patient may have weaned down to room air having a current FiO2 value (FiO2_{CURRENT}) of 21%. The algorithms may place boundaries on each of the factors that provides a lower limit of zero for each of the factors such that a negative numerator is not used in calculating the TVS value. Additionally or alternatively, when a negative value is calculated for a factor, the system may cause a prompt to be displayed for lowering the set negative value. The prompt may include a suggested adjusted minimum value and/or additional information to guide the clinician to select an adjusted minimum value.

A current support indicator 316 may also be included in the patient progress section 304. The current support indicator 316 indicates a current level of the total ventilator support being provided to the patient by the ventilator. The total ventilator support may be calculated or determined as discussed above. The current support indicator 316 may be a bar indicator as shown in FIG. 3A. A filled portion 318 of the current support indicator 316 indicates the current support level. For instance, an unfilled bar indicates minimum/minimal ventilator support and a fully filled bar indicates full ventilator support.

The family dashboard 302A may also include a patient’s breathing section 320 that includes indicia or data about the patient’s current breathing. The patient’s breathing section 320 includes a breathing section title 322 (e.g., John’s Breathing). The patient’s breathing section 320 may also include an independent breathing indicator 324. The independent breathing indicator 324 indicates the portion of breaths that are being triggered by the patient (e.g., spontaneous breaths) and the portion of breaths that being provided by the ventilator as mandatory breaths. For example, the independent breathing indicator 324 may indicate the percentage of spontaneous breaths and the percentage of mandatory breaths delivered by the ventilator over a period of time (e.g., the prior hour, prior day, etc.). The patient portion indicator 326 may include a patient portion indicator 326 (e.g., a bar) that indicates the percentage of spontaneous breaths over the time period and a ventilator portion indicator 328 (e.g., a bar) that indicates the percentage of mandatory breaths over the time period. A larger patient portion indicator 326 indicates a higher percentage of spontaneous breaths. Similarly, a larger ventilator portion indicator 328 indicates a higher percentage of mandatory breaths.

The family dashboard 302A may also include a patient’s strength section 330 that includes indicia or data about the patient’s strength. The patient’s strength section 330 also includes a strength section title 332 (e.g., John’s Strength). The patient’s strength section 330 may include a breathing muscle strength plot 334 that indicates the breathing muscle strength of the ventilated patient. The x-axis of the breathing muscle strength plot 334 may represent time and the y-axis may represent the breathing muscle strength value or amount. The plot 334 may include a trend line 336 indicating the trend of the breathing muscle strength of the patient over time. The breathing muscle strength of the patient may be equivalent to Pₘᵤₛ, which is the pressure generated by the respiratory muscles. In some examples, Pₘᵤₛ may be measured directly by a sensor attached to the patient. In other examples, Pₘᵤₛ may be calculated from measured properties or characteristics of the breathing gases.

Patient help suggestions 338 may also be displayed in the family dashboard 302A. The patient help suggestions 338 may include suggestions for the patient and/or the patient’s family members. In some examples, the patient help suggestions 338 may be static. In other examples, the patient help suggestions 338 may be dynamic. For instance, the patient help suggestions 338 may update based on the current condition of the patient. As an example, a first set of suggestions may be presented when the total ventilator support is above a support threshold, and a second set of suggestions may be presented when the total ventilator support is below the support threshold. Other metrics, such as the independent breathing of the patient and/or the muscle strength, may be used additionally or alternatively for triggering different sets of patient help suggestions 338 for display within the family dashboard 302A. In addition, time on the ventilator may also be used as a triggering condition for updating the patient help suggestions 338. For instance, for a first number of days (e.g., 5 days), a first set of suggestions 338 may be displayed, for a second number of days (e.g., days 6-10), a second set of suggestions 338 may be displayed, and for a third number of days (e.g., days 11-16), a third set of suggestions 338 may be displayed.

In addition, the patient help suggestions 338 may include a website location (e.g., uniform resource locator (URL)) and/or a scannable code (e.g., Quick Response (QR) code). The URL or QR code may be directed to a website with additional information relevant to the patient help suggestions 338 and/or the patient’s condition. For instance, a patient or family member may capture the QR code on his or her mobile device, which causes the corresponding website/webpage to load. By directing the patient or family member to a website, the corresponding information may be more robust than what can be displayed on the ventilator display 300. Moreover, redirecting to the website reduces the need or possibility of having the patient or family member directly interact (e.g., touch) the ventilator or ventilator screen. As an additional benefit, the website may be independently updated without having to update the ventilator software. Such independent updatability may be beneficial where data or information regarding a patient’s condition is rapidly changing, such as in the case of the COVID-19 disease caused by the SARS-CoV-2 virus. The URL and/or QR code may also be updated based on the same types of triggering conditions discussed above so that the patient and/or patient’s family may be directed to relevant information as the patient’s condition changes. For instance, the URL or QR code may direct the patient to different websites/webpages based on the triggering conditions and/or changing patient conditions discussed above.

A configuration lock 340 may also be included in the family dashboard 302A. The configuration lock 340 may be selectable to allow for reconfiguration of the dashboard 302A by a clinician. For instance, when a selection of the configuration lock 340 is received, a prompt for credentials may be displayed. The prompt for credentials may include input boxes for a pin, password, or other type of credentials used to authorize and authenticate a clinician.

When recognized credentials are entered into the prompt, the family dashboard 302A may enter a configuration mode that allows the clinician to reconfigure family dashboard 302A. While in the configuration mode, the configuration lock 340 may appear in an unlocked position. In addition, while in the configuration mode, the clinician may resize and/or rearrange the user interface elements and/or sections of the family dashboard 302A. The clinician may also choose to delete or add user interface components and/or sections. The rearrangement and/or resizing may be accomplished by drag-and-drop interactions or other suitable user interactions with the family dashboard 302A. For instance, when a user interface element is selected, resizing edges may be displayed and an “x” indicator may be displayed to remove or delete the element from the family dashboard 302A. An add-element indicator (e.g., a “+” indicator) may also be displayed, and upon selection, a list of additional user interface elements and/or sections may be displayed. Selection of one of the displayed elements and/or sections causes the selected element and/or sections to be added to the family dashboard. In some examples, text or other features of the user interface elements may be editable when in the configuration mode. For instance, the section titles may be edited to reflect the current patient’s name, the definitions of full/minimum support may be adjusted, etc.

Different preset/preconfigured dashboards or collections of user interface elements for different types of patients and/or conditions of patients may be stored or otherwise available for use. As an example, the preconfigured dashboards may include an adult dashboard, a pediatric dashboard, a COVID dashboard, a clinician dashboard, etc. For instance, a pediatric dashboard may provide indicators that are more appropriate for a pediatric or neonatal patient, such as a trend of the patient’s weight. Similarly, a clinician dashboard may provide more detail than the standard adult patient, and such a clinician dashboard may be useful for clinicians during rounds or when evaluating the patient. Accordingly, a default view of the ventilator may include the most detailed or complex information, such as ventilator waveforms, a clinician dashboard may include a medium amount of complexity or detail (e.g., less detailed or less complex than the default view), and a family dashboard may include the least complex or detailed information (e.g., less complex or detailed than the clinician dashboard).

FIG. 3B depicts additional or alternative user-interface elements for the patient or family dashboard 302B. The example family dashboard 302B is a simplified dashboard that may be appropriate for an adult patient. The family dashboard 302A may include the patient name 352 and a photo or image of the patient 354. The patient image 354 may be uploaded when ventilation begins and/or when the family dashboard 302B is configured. In other examples, the patient image 354 may be uploaded remotely from a remote device, such as a mobile phone, of the patient and/or the patient’s family.

Clinician contact information 356 may also be included in the family dashboard 302B. The clinician contact information 356 may include the name of a clinician (doctor, nurse, etc.) and the corresponding contact information for that clinician, such as the clinician’s phone number or other means of contact. A clinician image 358 for the clinician may also be displayed so that the patient and/or the patient’s family may be able to more easily recognize the clinician when the clinician enters the room.

A percent support indicator 360 may also be incorporated in the family dashboard 302B. The percent support indicator 360 may indicate the current percent of total ventilator support and may be calculated in the ways discussed above. The percent support indicator 360 may display a number as shown in FIG. 3B. In other examples, the percent support indicator 360 may be a geometric or bar indicator that conveys the total ventilator support.

The family dashboard 302B may also include a real-time breathing indicator 362. The real-time breathing indicator 362 indicates a live representation of the patient’s breathing. For instance, the real-time breathing indicator 362 may be animated or move as the patient inhales and exhales breathing gases. In the example depicted in FIG. 3B, the real-time breathing indicator 362 is a bar with a filled portion 363 that grows and shrinks with the inhalation and exhalation of the patient. As an example, during peak inhalation of the patient, the filled portion 363 is the largest (e.g., fills the largest portion of the bar), and during exhalation, the filled portion 363 is the smallest (e.g., fills the smallest portion of the bar).

FIG. 3C depicts additional or alternative user-interface elements for the patient or family dashboard 302C. Similar to the example family dashboard 302B, the example family dashboard 302C is also further simplified from the family dashboard 302A in FIG. 3A. The example family dashboard 302C may be appropriate for a pediatric or neonatal patient. Similar to the family dashboard 302B, the family dashboard 302C includes a patient name 352, a patient image 354, clinician contact information 356, and a clinician image 358.

The family dashboard 302C may also include an animated lung-shaped breathing indicator 368. The animated lung-shaped breathing indicator 368 operates similarly to the real-time breathing indicator 362 discussed above. For instance, a filled portion 369 of the animated lung-shaped breathing indicator 368 may change as the patient breathes. As an example, the filled portion 369 may enlarge as the patient inhales, and the filled portion 369 may shrink when the patient exhales. In other examples, the filled portion 369 may be omitted and the shape or size of the animated lungs may change based on the patient’s breathing. For example, the display of the lungs may expand when the patient inhales and may contract when the patient exhales.

A patient weight plot 364 may also be included in the family dashboard 302C. The x-axis of the patient weight plot 364 represents time, and the y-axis of the patient weight plot 364 represents the weight of the patient. The patient weight plot 364 also includes a trend line 366 of the patient’s weight. The patient weight plot 364 may be particularly useful for neonatal patients because the relative weight changes are larger and such growth is often indicative of positive neonatal progress. The weight plot 364 may alternatively be a growth or length plot that indicates the growth and/or length trend of the patient as well.

The plots described above may also include indicators for average times that patients with the current patient’s condition are typically on ventilation. For instance, a vertical line or indicator may be displayed in the plot to show the average time on ventilation for a particular condition. Different standard deviations or other statistics based on the average times may also be displayed. For instance, a first indicator for the 25^{th} percentile of ventilation time, a second indicator for the 50^{th} percentile of ventilation time, and a third indicator for the 75^{th} percentile of ventilation time. The average or percentile indicators may help a patient or family member assess how the patient is progressing compared to that of an average patient.

The above indicators and/or user interface elements may be combined in different manners than shown above. Additional indicators not explicitly shown in the figures or discussed above may also be incorporated in the dashboards of the present technology. For example, an oxygen saturation indicator that indicates a current patient oxygen saturation (e.g., SpO₂) or an oxygen saturation trend may be included. Indicators for other physiologic parameters of the patient, such as heart rate, respiration rate, etc. may also be included. A calendar or indication of the number of days the patient has been ventilated may also be included (and/or the number of days since admission, intubation, surgery, etc.). Indicators about the specific ventilation settings may also be included, such as a breath mode indicator indicating the breath mode of the ventilator, a patient interface indicator indicating the type of patient interface connected to the patient (e.g., endotracheal tube, mask, cannula), a PAV+ support percentage indicator indicating a current support percentage or a trend of support percentage for a proportional assist ventilation (PAV) mode.

In some examples, the user interface elements of the family dashboard(s) 302 may be selectable to further drill down into the data underlying the respective user interface elements. As an example, selection of the total ventilator support plot 308 may cause the underlying data used to calculate the total ventilator support to be displayed on the display 300. For instance, the minute volume, minute volume from spontaneous breaths, the PIP, etc. may be displayed when the total ventilator support plot 308 is selected. As another example, selecting one of the elements may cause the display of pressure and oxygenation settings.

While the family dashboards discussed above are shown as displayed on the display 300 of ventilator, the family dashboards may also be displayed on other screens or devices. For instance, the family dashboards may be replicated on a remote computing device, such as a laptop, tablet, or mobile device of a family member or of the hospital. Accordingly, a family member may be able to view the dashboard(s) outside of the patient’s room, which may be particularly useful for patients in the neonatal intensive care unit (NICU) and/or patients with highly infectious diseases. For example, a family member may already have a tablet, and an app for the family ventilator dashboard may be downloaded onto the tablet to view the tablets discussed herein. In some examples, the family dashboard may also be accessed via a web address from a web browser of the remote device. The transmission of the family dashboard (and/or the underlying ventilation data) may be initiated based on an interaction with the ventilator. For instance, a clinician may need to select a setting or a user interface element that allows for, or causes, the transmission of the family dashboard to the remote device. Accordingly, additional security is provided to keep the family dashboards on the ventilator until sharing of that dashboard is authorized by the clinician.

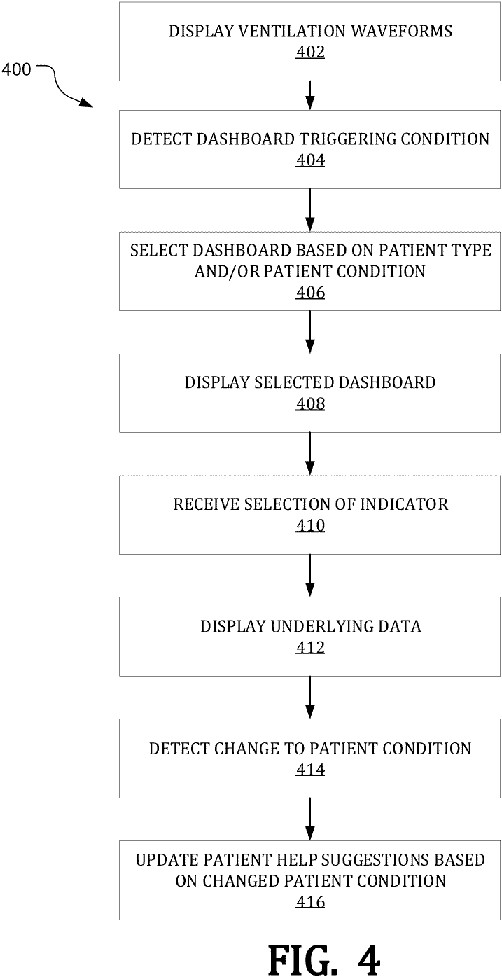

FIG. 4 depicts an example method 400 for generating a family dashboard. The operations of method 400 may be performed by any of the components discussed herein, such as the ventilator and/or the components thereof. At operation 402, ventilation waveforms are displayed on a display of the ventilator. The ventilation waveforms may form a part of the default or standard view of the ventilator that displays complex data intended for the well-trained medical professionals.

At operation 404, a dashboard triggering condition is detected. The dashboard triggering condition may include an expiration of a timeout timer or a selection of a dashboard activation element. The dashboard activation element may be a selectable user interface element presented on the display. In other examples, the dashboard activation element may be a selectable physical element, such as a button or a switch.

Based on the detection of the dashboard triggering condition, the dashboard is selected based on a type of the patient and/or a condition of the patient at operation 406. For instance, the ventilator may store a plurality of a preconfigured dashboards that are configured for different types of patients and/or patients with certain conditions. For instance, preconfigured dashboards may include an adult dashboard, a pediatric dashboard, a COVID dashboard, etc.

At operation 408, the selected dashboard is displayed on the display of the ventilator. The user interface elements of the selected dashboard are populated with the data specific to the patient. For instance, a total ventilation support plot may be populated with the data for the patient being ventilated by the ventilator. The data specific to the patient may be generated or derived based on physiological parameters of the patient and/or characteristics of the breathing gases delivered by the ventilator. Such parameters and/or characteristics may be measured by sensors or the ventilator and/or sensors in communication with the ventilator. In some examples, the data specific to the ventilated patient may be generated or derived from the ventilation settings of the ventilator, such as oxygenation settings, pressure settings, flow settings, PEEP settings, etc.

The user interface elements of the displayed dashboard may include any of the user interface elements discussed above. For example, the dashboard may include two, three, four, five or more of: a total ventilator support indicator that indicates amount of ventilator support being provided to the patient; an independent breathing indicator that indicates the portion of breaths that are being triggered by the patient; a breathing muscle strength plot that indicates a breathing muscle strength of the ventilated patient; patient help suggestions; a scannable code configured to direct a scanning device to a webpage with additional information regarding a condition of the patient; clinician contact information; a real-time breathing indicator; or a patient weight plot.

At operation 410, a selection of one of the indicators of the displayed dashboard is received. Based on receiving the selection in operation 410, underlying data used to populate the selected indicator is displayed in operation 412. For example, if the total ventilator support plot is selected, the data used to calculate the total ventilator support plot may be displayed. The underlying data may be displayed as an overlay over at least a portion of the selected indicator. In other examples, the underlying data may replace the display of the selected indicator.

At operation 414, a change in the patient condition is detected, and in response, patient help suggestions and/or a URL or QR code of the dashboard are updated at operation 416. For instance, as discussed above, the suggestions, URL, and/or QR code may change based on the condition of the patient, such as how long the patient has been ventilated and/or the current total ventilation support for the patient.

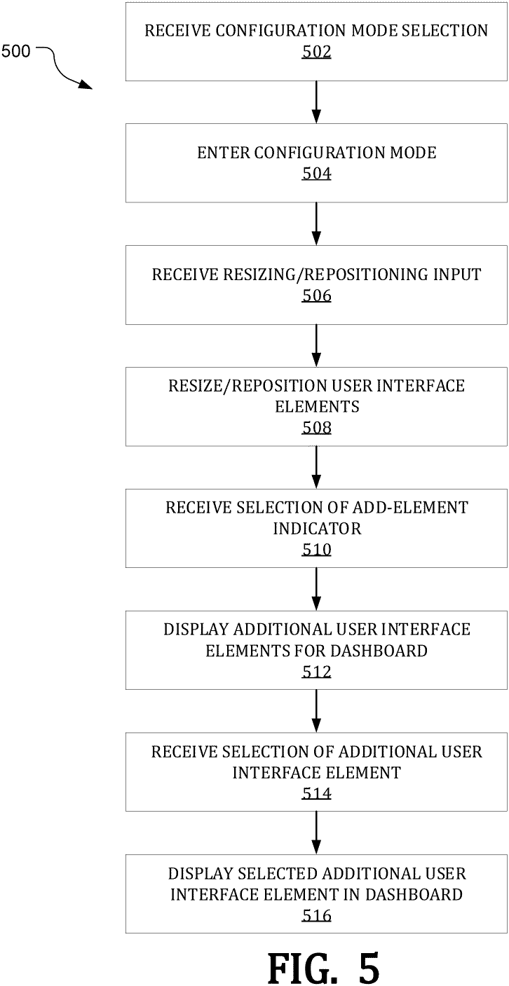

FIG. 5 depicts another example method 500 for configuring a patient or family dashboard. The operations of method 500 may be performed by any of the components discussed herein, such as the ventilator and/or the components thereof. At operation 502, a selection to enter a configuration mode is received. The selection may be a selection of a configuration lock or similar user interface element. In response to the selection received in operation 502, a configuration mode is entered at operation 504. Entering the configuration mode may include altering the display to indicate that the ventilator is in the configuration mode, such as by changing color schemes, displaying “Configuration Mode,” showing a configuration lock in an unlocked position, etc.

While in the configuration mode, input to resize or reposition one or more of the user interface elements in the dashboard is received at operation 506. The input may be drag-and-drop input or other types of input. Based on received input, the corresponding user interface element(s) are resized and/or repositioned in the displayed dashboard at operation 508.

Also while in the configuration mode, a selection of an add-element indicator is received at operation 510. The add-element indicator may be a “+” user interface element or other similar type of selectable element. Based on the received selection, additional user interface elements (e.g., indicators) that could potentially be included in the dashboard are displayed at operation 512. The additional user interface elements may include any of the elements discussed above that are not already included in the displayed dashboard. At operation 514, a selection of one of the displayed additional user interface elements is received, and at operation 516, the selected additional user interface element is displayed in the dashboard.

Operations 506-516 may repeat as necessary while the clinician configures the particular dashboard with the types of desired indicators as well as the layout for the desired indicators. Once the dashboard is configured to the satisfaction of the clinician, the dashboard may be saved for immediate display or later display. The dashboard may be saved as one of a set of dashboards, and the dashboard may be saved with a title or label such that it is identifiable from other dashboards in the set. The clinician may also configure the patient type and/or patient condition for the dashboard, such that the dashboard may later be automatically retrieved when a patient having that patient type and/or patient condition is connected to the ventilator.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A ventilator for generating a family dashboard for a patient being ventilated by the ventilator, the ventilator comprising:
a pressure generating system for generating pressurized breathing gases for delivery to the patient;
a display coupled to the pressure generating system;
a processor; and
memory storing instructions that, when executed by the processor, cause the ventilator to perform operations comprising:
displaying, on the display, a set of ventilation waveforms;
detecting a triggering condition for a family dashboard; and
based on detecting the triggering condition, removing the ventilation waveforms from the display and displaying, on the display, the family dashboard, wherein the family dashboard includes at least a total ventilator support indicator that indicates an amount of total ventilator support being provided to the patient.

2. The ventilator of claim 1, wherein the triggering condition is one of an expiration of a timeout timer or a selection of a family dashboard activation element.

3. The ventilator of claim 1, wherein the operations further comprise, based on at least one of a patient type or patient condition, selecting the family dashboard from a set of preconfigured family dashboards.

4. The ventilator of claim 1, wherein the total ventilator support indicator is a total ventilator support plot with an x-axis representing time and a y-axis representing the amount of total ventilator support.

5. The ventilator of claim 1, wherein the amount of total ventilator support is calculated from at least a spontaneous breathing factor and a pressure factor.

6. The ventilator of claim 5, wherein the amount of total ventilator support is further calculated from at least an oxygenation factor.

7. The ventilator of claim 1, wherein the amount of total ventilator support is a percentage ranging from 0% to 100% and is based on at least a total minute volume, a spontaneous minute volume, a peak inspiratory pressure, and an oxygenation factor.

8. The ventilator of claim 1, wherein the total ventilator support indicator is a current ventilator support indicator.

9. The ventilator of claim 1, wherein the family dashboard further includes at least one of:
an independent breathing indicator that indicates a portion of breaths that are being triggered by the patient; or
a breathing muscle strength plot that indicates a breathing muscle strength of the patient.

10. The ventilator of claim 1, the family dashboard further includes a scannable code configured to direct a scanning device to a webpage with additional information regarding a condition of the patient.

11. A method for generating a family dashboard for a ventilated patient, the method comprising:
displaying, on a display of a ventilator, one or more ventilation waveforms;
detecting a triggering condition for a family dashboard;
based on detecting the triggering condition, removing the ventilation waveforms from the display and displaying, on the display, the family dashboard, wherein the family dashboard includes at least three user interface elements selected from:
a total ventilator support indicator that indicates an amount of ventilator support being provided to the patient;
an independent breathing indicator that indicates a portion of breaths that are being triggered by the patient;
a breathing muscle strength plot that indicates a breathing muscle strength of the patient;
patient help suggestions;
a scannable code configured to direct a scanning device to a webpage with additional information regarding a condition of the patient;
clinician contact information;
a real-time breathing indicator; and
a patient weight plot.

12. The method of claim 11, wherein the family dashboard includes the total ventilator support indicator, the independent breathing indicator, and the breathing muscle strength plot.

13. The method of claim 11, further comprising:
receiving a selection to enter a configuration mode;
entering the configuration mode;
while in the configuration mode, receiving an input to resize or reposition one user interface element of the user interface elements in the family dashboard; and
based on the received input, resizing or repositioning the user interface element.

14. The method of claim 11, further comprising:
receiving a selection to enter a configuration mode;
entering the configuration mode;
while in the configuration mode, receiving a selection of an add-element indicator;
in response to receiving the selection of the add-element indicator, presenting additional user interface elements for adding to the family dashboard;
receiving a selection of one of the additional user interface elements; and
displaying the selected additional user interface element in the family dashboard.

15. The method of claim 11, further comprising:
receiving a selection of one of the user interface elements of the family dashboard; and
in response to receiving the selection, displaying underlying data used to generate the user interface element.

16. A method for generating a family dashboard for a ventilated patient, the method comprising:
detecting, by a ventilator, a triggering condition to display a family dashboard;
based on at least one of a patient type or a patient condition, selecting, by the ventilator, a family dashboard from a set of preconfigured family dashboards; and
displaying, on a display of the ventilator, the selected family dashboard.

17. The method of claim 16, wherein the patient type is pediatric and the selected family dashboard is a pediatric dashboard that includes a patient weight plot that indicates a weight trend of the patient.

18. The method of claim 17, wherein the pediatric dashboard further includes an animated lung-shaped breathing indicator.

19. The method of claim 17, wherein the pediatric dashboard further includes clinician contact information and a clinician image.

20. The method of claim 17, wherein the pediatric dashboard further includes a scannable code configured to direct a scanning device to a webpage with additional information regarding a condition of the patient.
